(19) United States
(12) Patent Application Publication
(10) Pub. No.: US 2026/0183148 A1
(43) Pub. Date: Jul. 2, 2026

(54) Automated transscleral cyclophotocoagulation

(71) Applicant: Belkin Vision Ltd., Yavne (IL)

(72) Inventors: Zachary Shane Sacks, Modiin (IL); Michael Belkin, Kiryat Ono (IL)

(21) Appl. No.: 19/130,134

(22) PCT Filed: Nov. 14, 2023
(86) PCT No.: PCT/IB2023/061472

§ 371 Date: May 15, 2025

Related U.S. Application Data
(60) Provisional application No. 63/432,714, filed on Dec. 15, 2022.

(51) Int. Cl. A61F 9/008; A61F 9/009

(52) U.S. Cl. CPC A61F 9/00823; A61F 9/009; A61F 2009/00844; A61F 2009/00865; A61F 2009/00868; A61F 2009/00897

(57) ABSTRACT

Apparatus (100, 400, 500) for medical treatment includes a contact lens (150, 302, 308, 602) having a front side (151) and a concave rear side (153), which is shaped to contact a front surface (155) of an eye (142) and comprises multiple protrusions (162, 304, 310) extending from the rear side and configured to contact and depress respective points around a sclera (168) of the eye. A laser (108) generates a beam (113) of optical radiation. A scanner (110) directs the beam through the front side of the contact lens to impinge sequentially on at least some of the protrusions so as to photocoagulate tissue in the eye in proximity to the respective points.

### CROSS-REFERENCE TO RELATED APPLICATION

This application claims the benefit of U.S. Provisional Patent Application 63/432,714, filed Dec. 15, 2022, which is incorporated herein by reference.

### FIELD OF THE INVENTION

The present invention relates generally to apparatuses, devices, and methods for treatment of the eye, and particularly to ophthalmic laser surgery.

### BACKGROUND

In a transscleral cyclophotocoagulation (TSCPC) procedure, the ciliary body of an eye is irradiated through the sclera, resulting in heating and consequent photocoagulation of the ciliary body. The coagulation of the ciliary body decreases the production of aqueous humor and thereby lowers the pressure in the eye.

### SUMMARY

Embodiments of the present invention that are described hereinbelow provide improved apparatuses, devices, and methods for ophthalmic laser surgery.

There is therefore provided, in accordance with an embodiment of the invention, a device for ophthalmic surgery, including a transparent material having a concave rear side, which is shaped to contact a front surface of an eye. Multiple protrusions extending from the rear side are distributed azimuthally around the rear side.

In the disclosed embodiments, the protrusions are configured to contact and depress respective points around a sclera of the eye. In one embodiment, multiple through-holes extend through the transparent material in respective proximity to the multiple protrusions.

In example embodiments, the protrusions have a dome shape or a rectangular profile. Typically, the protrusions have a height of at least 0.1 mm or even at least 0.5 mm. Additionally or alternatively, the protrusions have a diameter of at least 0.4 mm or even at least 0.8 mm.

In some embodiments, the protrusions are distributed on a circle having a diameter between 12.5 and 15 mm. The protrusions may be distributed around an entire circumference of the circle or around a part of a circumference of the circle, with one or more gaps in the circumference in which there are no protrusions.

There is also provided, in accordance with an embodiment of the invention, apparatus for medical treatment, including a contact lens having a front side and a concave rear side, which is shaped to contact a front surface of an eye and includes multiple protrusions extending from the rear side and configured to contact and depress respective points around a sclera of the eye. A laser is configured to generate a beam of optical radiation. A scanner is configured to direct the beam through the front side of the contact lens to impinge sequentially on at least some of the protrusions so as to photocoagulate tissue in the eye in proximity to the respective points.

In some embodiments, the apparatus includes a camera configured to capture an image of the contact lens in contact with the front surface of the eye and a controller, which is configured to process the image to identify the protrusions and to control the scanner to direct the beam to impinge on the protrusions. The apparatus may further include a sensor, which is configured to sense a temperature of the eye at each of the points contacted by the protrusions, wherein the controller is configured to control a dose of energy applied by the laser at each of the points responsively to the temperature. In disclosed embodiments, the sensor is configured to sense infrared radiation emitted from the points.

In some embodiments, the apparatus includes an enclosure, which contains the scanner, the camera, and the sensor and which includes a window through which the beam of optical radiation is directed toward the contact lens and through which the camera and the sensor receive the image and the infrared radiation from the eye, wherein the window is transparent in a range between 400 nm and at least 5000 nm. In one embodiment, the apparatus includes a dichroic beamsplitter, which directs visible radiation emitted from the eye toward the camera and directs the infrared radiation emitted from the eye toward the sensor.

In a disclosed embodiment, the sensor includes a thermal camera, which is configured to capture an infrared image of the eye. Additionally or alternatively, the contact lens has multiple through-holes extending therethrough in respective proximity to the multiple protrusions, wherein the sensor is configured to sense the infrared radiation that passes through the through-holes.

In an alternative embodiment, the apparatus includes a microphone coupled to the controller and configured to receive acoustic signals from the eye while the beam is photocoagulating the tissue, wherein the controller is configured to control a dose of energy applied by the laser at each of the points responsively to the acoustic signals.

Additionally or alternatively, the apparatus includes an eye-stabilizing structure, coupled between the contact lens and an optical unit containing the scanner.

There is additionally provided, in accordance with an embodiment of the invention, a method for medical treatment, which includes providing a contact lens having a front side and a concave rear side, which is shaped to contact a front surface of an eye and includes multiple protrusions extending from the rear side. The contact lens is applied to the eye so that the protrusions contact and depress respective points around a sclera of the eye. A beam of optical radiation is directed from a laser through the front side of the contact lens to impinge sequentially on at least some of the protrusions so as to photocoagulate tissue in the eye in proximity to the respective points.

In a disclosed embodiment, directing the beam includes applying transscleral cyclophotocoagulation to a ciliary body in the eye.

There is further provided, in accordance with an embodiment of the invention, apparatus for medical treatment, including a laser configured to generate a beam of optical radiation and a scanner, which is configured to direct the beam toward points on an outer surface of an eye under treatment. A camera is configured to capture images of the eye during the treatment. A sensor is configured to sense a temperature of the outer surface of the eye. A controller is configured to process the images to identify multiple treatment points on a sclera of the eye around an outer edge of a limbus of the eye, to control the scanner to direct the beam to impinge sequentially on the identified treatment points so as to photocoagulate tissue in the eye in proximity to each of the identified treatment points, and to control a dose of energy applied by the laser at each of the points responsively to the temperature.

In some embodiments, the apparatus includes an enclosure, which contains at least the scanner, the camera, and the sensor, and a window in the enclosure, through which the beam is directed toward the eye by the scanner and through which the camera and the sensor receive the images and the infrared radiation from the eye. In a disclosed embodiment, the apparatus includes a fixation point, which is contained in the enclosure and is visible to the eye through the window.

In one embodiment, the controller is configured to direct the beam to impinge on the identified treatment points without contact between any part of the apparatus and the eye. In other embodiments, the apparatus includes a contact lens, wherein the controller is configured to direct the beam to impinge on the identified treatment points through the contact lens while the contact lens is applied to the eye.

There is moreover provided, in accordance with an embodiment of the invention, a method for medical treatment, which includes capturing images of an eye under treatment and sensing a temperature of an outer surface of the eye. The images are processed to identify multiple treatment points on a sclera of the eye around an outer edge of a limbus of the eye. A beam of optical radiation from a laser is directed to impinge sequentially on the identified treatment points so as to photocoagulate tissue in the eye in proximity to each of the identified treatment points. A dose of energy applied by the laser at each of the points is controlled responsively to the temperature.

In some embodiments, directing the beam includes applying transscleral cyclophotocoagulation to a ciliary body in the eye. Applying the transscleral cyclophotocoagulation may include directing the beam to impinge on the identified treatment points without contacting the eye. Alternatively, applying the transscleral cyclophotocoagulation includes directing the beam to impinge on the identified treatment points through a contact lens that is applied to the eye.

The present invention will be more fully understood from the following detailed description of the embodiments thereof, taken together with the drawings in which:

### BRIEF DESCRIPTION OF THE DRAWINGS

FIG. 1 is a schematic side view of an automated TSCPC apparatus, in accordance with an embodiment of the invention;

FIGS. 2A, 2B, and 2C are schematic frontal views of contact lenses placed on an eye, in accordance with embodiments of the invention;

FIGS. 3A and 3B are schematic frontal and detail views of contact lenses, in accordance with embodiments of the invention;

FIG. 4 is a schematic side view of an automated TSCPC apparatus, in accordance with an additional embodiment of the invention;

FIG. 5 is a schematic side view of an automated TSCPC apparatus, in accordance with another embodiment of the invention;

FIG. 6 is a schematic frontal view of a contact lens with protrusions and through-holes, in accordance with an embodiment of the invention; and

FIG. 7 is a flowchart that schematically illustrates a method for performing a TSCPC procedure, in accordance with an embodiment of the invention.

### DETAILED DESCRIPTION OF EMBODIMENTS

### Overview

In a typical TSCPC procedure, a patient is either seated or lying down proximate to a TSCPC apparatus or a hand piece of the apparatus, which is aligned with the patient's eye. The patient must remain with his/her head in a fixed position and orientation with respect to the TSCPC apparatus or the handpiece while a laser beam is focused on and shifted across the ciliary body by an operator, such as an ophthalmologist.

As a large number of laser pulses are fired into the ciliary body with concomitant alignment of the laser beam for each pulse, the procedure may be prolonged, taxing the stamina of the operator performing the procedure, as well as the ability of the patient to keep his/her head in the proper position and orientation. Moreover, the procedure requires the operator to be skilled in accurate alignment of the laser beam on specific points on the ciliary body, as well as for setting the length and power of the laser exposure so as not to over-treat (over-expose) and damage the eye. Over-treating may be indicated by a popping sound in some cases, but in other cases a certain amount of popping is normal and provides feedback to the ophthalmologist administering the treatment. There is a need for feedback and automation to control the dosage and duration of treatment.

In response to this need, embodiments of the present invention provide apparatus and methods that automate and shorten the duration of the TSCPC procedure while ensuring that its efficacy and safety are maintained. Some of these embodiments use a novel contact lens, which is placed on the eye of the patient during the procedure. The contact lens is used in conjunction with an optical unit comprising a laser, a scanner, a camera, and a controller. The rear side of the contact lens, which contacts the front surface of the eye, has multiple transparent protrusions around the perilimbal sclera. The protrusions contact and depress the conjunctiva and sclera of the eye so as to remove water and blood from the conjunctiva and sclera at the target points for irradiation by the laser. The camera captures an image of the eye, including the contact lens, and the controller of the optical unit then directs the laser beam through some or all of the protrusions into the ciliary body. The removal of water and blood from the conjunctiva and sclera at the protrusions increases the transmission of the laser beam into the ciliary body and thus enhances the efficacy of the treatment.

The treatment plan, i.e., the selection of the protrusions through which the laser beam is to be directed, can be chosen by the operator or chosen automatically before the actual laser irradiation of the eye. During the irradiation the controller sets the direction of the laser beam automatically with only minimal delays between treatment points. The contact lens, which has been aligned with the eye before beginning the treatment, ensures that the laser beam will irradiate the proper target points. Thus, the time required to complete the treatment is significantly reduced in comparison with manual procedures, in which the operator directs the laser from treatment point to treatment point. This, in turn, reduces the strain both on the operator and on the patient.

In some embodiments, the optical unit comprises a thermal camera or detector to acquire thermal images of the eye, and thus provide feedback for controlling the dosing of the laser. When the contact lens is made from a material that is not transparent to the infrared (IR) radiation used for temperature monitoring, the contact lens may have multiple through-holes adjacent to the protrusions to facilitate thermal sensing of the tissue surface.

The disclosed embodiments thus provide a device for ophthalmic surgery, specifically a contact lens, comprising a transparent material having a concave rear side, which is shaped to contact a front surface of an eye, and multiple protrusions extending from the rear side and distributed azimuthally around the rear side. The protrusions contact and depress respective points around the sclera of the eye. A laser generates a beam of optical radiation, and a scanner directs the beam through the front side of the contact lens to impinge sequentially on at least some of the protrusions so as to photocoagulate tissue in the eye in proximity to the respective points.

Although the disclosed embodiments relate specifically to TSCPC, the principles of the present invention, including special-purpose contact lenses with protrusions that depress the front surface of the eye, may also be applied, mutatis mutandis, in other ophthalmic treatment procedures. These procedures may include procedures to alter the mechanical properties of the sclera to make it stronger or weaker or to increase fluid permeability for treating presbyopia or glaucoma.

### System Description

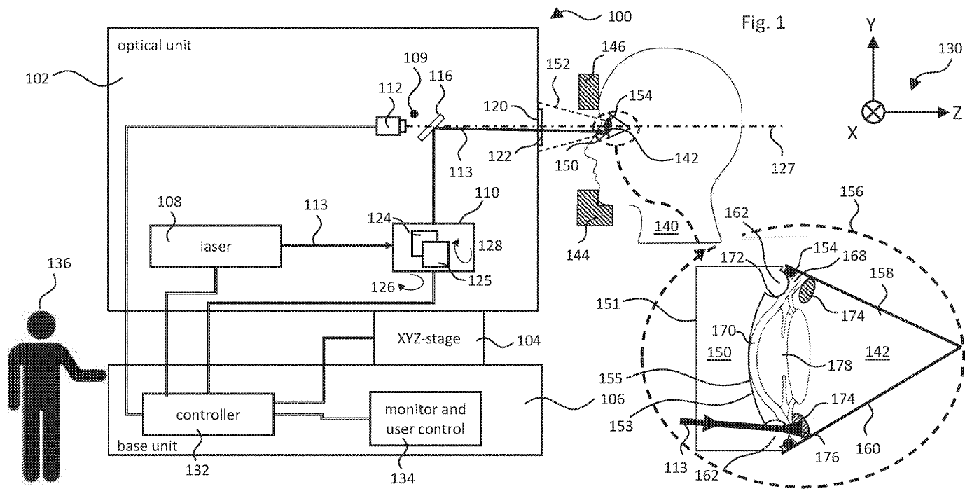

FIG. 1 is a schematic side view of an automated TSCPC apparatus 100, in accordance with an embodiment of the invention.

TSCPC apparatus 100 comprises an optical unit 102, an XYZ-stage 104, and a base unit 106. Optical unit 102 comprises a treatment laser 108 emitting a treatment beam 113 of optical radiation. The optical unit also comprises a scanner 110, a camera 112, a fixation point 109, and a beam combiner 116, which combines the optical paths of laser 108 and camera 112.

In the present embodiment, laser 108 comprises one or more laser diodes, which emit optical radiation in the near IR (NIR) range, for example at a wavelength of 810 nm. The diode output may be coupled through an optical fiber, for example with a diameter of 400 μm. Lasers with high beam quality are advantageous in allowing scanner 110 to be located far from the eye to be treated, for example 200-300 mm away. Laser 108 may comprise a tapered diode with an optical amplifier, such as the Toptica EYP-TPA-0808-02000-4006-CMT04-0000 (manufactured by Toptica Photonics AG, Graefelfing, Germany). Alternatively, laser 108 may comprise an optically pumped semiconductor laser, spectrally beam-combined diode arrays, or a titanium sapphire laser, for example. Laser 108 is typically operated in a pulsed mode, with one or more pulses emitted for each irradiated spot during the TSCPC procedure.

Scanner 110 comprises two galvanometer mirrors 124 and 125 rotating around two orthogonal axes (not shown for the sake of simplicity), with the rotations indicated by respective circular arrows 126 and 128. Scanner 110 directs beam 113 through a contact lens 150 into an eye 142, as will be further detailed hereinbelow. In alternative embodiments, scanners of other types may be used, such as one or more rotating prisms or voice coil mirrors (for example, Optotune MR-15-30, manufactured by Optotune Switzerland AG, Dietikon, Switzerland).

XYZ-stage 104 moves optical unit 102 in three linear orthogonal X-, Y-, and Z-directions, as indicated by Cartesian coordinates 130, to align optical unit 102 with eye 142.

Base unit 106 comprises a controller 132, as well as a monitor and user control unit 134. Controller 132 receives inputs from and outputs control signals to camera 112, laser 108, scanner 110, XYZ-stage 104, and monitor and user control unit 134. Alternatively, the monitor and/or the entire user control unit 134 and/or the controller may be integrated in optical unit 102.

The functionality of controller 132 can be implemented in hardware logic, for example using one or more fixed-function or general-purpose integrated circuits, Application-Specific Integrated Circuits (ASICs), and/or Field-Programmable Gate Arrays (FPGAs). Alternatively or additionally, controller 132 may perform at least some of the functionality described herein by executing software and/or firmware code. For example, controller 132 may comprise a programmable processor comprising, for example, a central processing unit (CPU) and/or a Graphics Processing Unit (GPU). Program code, including software programs and/or data may be loaded for execution and processing by the CPU and/or GPU. The program code and/or data may be downloaded to the controller in electronic form over a network, for example. Alternatively or additionally, the program code and/or data may be provided and/or stored on non-transitory tangible media, such as magnetic, optical, or electronic memory. Such program code and/or data, when provided to the controller, produce a machine or special-purpose computer, configured to perform the tasks described herein.

Monitor and user control unit 134 comprises one or more visual displays and suitable input devices, such as a keyboard, joystick, and/or mouse, enabling an operator 136 to interact with TSCPC apparatus 100. (Details of monitor and user control unit 134 have been omitted from the figure for the sake of simplicity.)

For a TSCPC procedure, a patient positions his/her head 140 in front of optical unit 102, so that his/her eye 142 is in proximity to a distal face 120 of the optical unit. Apparatus 100 typically comprises a chin rest 144 and a forehead rest 146 for enhanced stability of patient's head 140 during the procedure. Chin rest 144 and forehead rest 146 may be integrated into base unit 106 or attached to a table of apparatus 100. The patient is instructed to gaze at fixation point 109 during the procedure.

Operator 136 observes eye 142 in an image captured by camera 112 and displayed on a monitor of unit 134. For this purpose, eye 142 may be illuminated by an illumination ring 122, for example, although alternatively, other sorts of light sources may be used. While observing the eye, operator 136 moves, using an input device such as a joystick, optical unit 102 in the X- and Y-directions so that an optical axis 127 of camera 112 is aligned with eye 142. During the TSCPC procedure, contact lens 150 is held against eye 142. Contact lens 150 may be held by the operator's hand or coupled to distal face 120, for example via an optional eye-stabilizing structure 152, which comprises struts and/or other mechanical elements extending between distal face 120 and contact lens 150. A suction ring 154 may be placed between the outer edge of contact lens 150 and eye 142 to facilitate keeping the lens in a stable contact with the eye. Prior to making contact, a gel or other suitable contact material may be applied to eye 142.

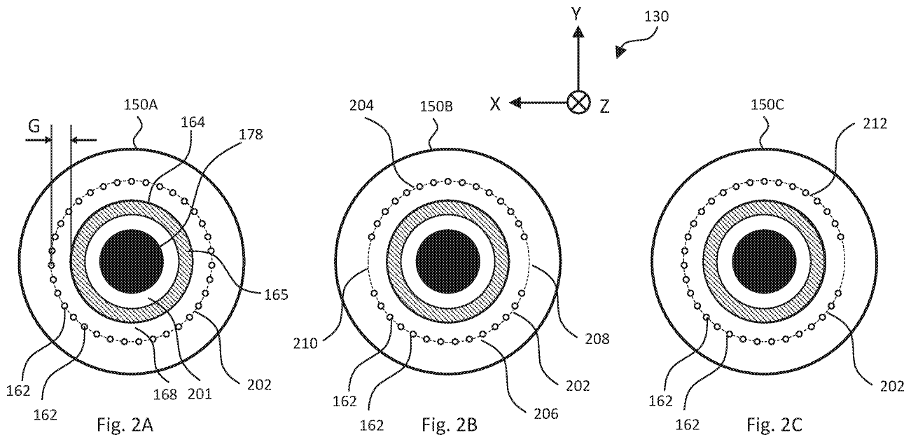

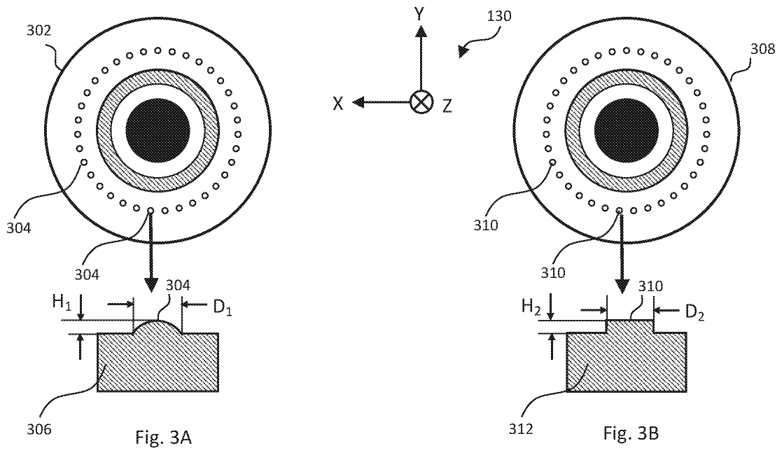

An inset 156 shows a sectional view of contact lens 150 and the frontal part of eye 142 between upper and lower eyelids 158 and 160. Contact lens 150 has a front side 151 (typically planar) and a concave rear side 153, and is shaped to contact a front surface 155 of eye 142. Contact lens 150 further has multiple transparent protrusions 162 extending from rear side 153 and distributed azimuthally around rear side 153 so as to contact corresponding points on front surface 155 of eye 142 and to push into the eye. Protrusions 162 are arranged to be aligned with a ciliary body 174 of eye 142. An outer edge 164 of a limbus 165 (hereinbelow also referred to as “outer limbal edge 164”) is used as a reference for this alignment with ciliary body 174, as illustrated in FIGS. 2A-2C hereinbelow. Operator 136 typically chooses a contact lens 150 from a kit of lenses such that the distance between protrusions 162 and outer limbal edge 164 is typically around 1.5 mm, and may vary between 0.5 mm and 2 mm. Protrusions 162, further detailed in FIGS. 2A-2C and 3A-3B hereinbelow, contact and depress a conjunctiva 172 and a sclera 168 of eye 142 so as to remove water and blood from the conjunctiva and sclera at potential target points and thus to increase the transmission of laser beam 113 into ciliary body 174 of eye 142.

Either before or after the alignment process described hereinabove and placing contact lens 150 against eye 142, operator 136 selects target points, i.e., the points corresponding to selected protrusions 162 where ciliary body 174 is to be irradiated. After verifying the alignment of contact lens 150 around outer limbal edge 164, operator 136 commands controller 132 to initiate the treatment sequence. In this sequence, camera 112 captures an image of contact lens 150. Controller 132 processes the captured image to identify protrusions 162 and controls scanner 110 to direct beam 113 through front side 151 to impinge sequentially to selected protrusions. After passing through a given protrusion 162, beam 113 is transmitted through and scattered by sclera 168 into ciliary body 174, as shown by an expanding beam 176 in inset 156.

During the procedure, controller 132 receives a stream of images from camera 132 and processes the images to track movement of eye 142 and/or contact lens 150 and to adjust scanner 110 accordingly. Typically, controller 132 controls scanner 110 so as not to aim at a pupil 178 of eye 142 even when no beam is being fired.

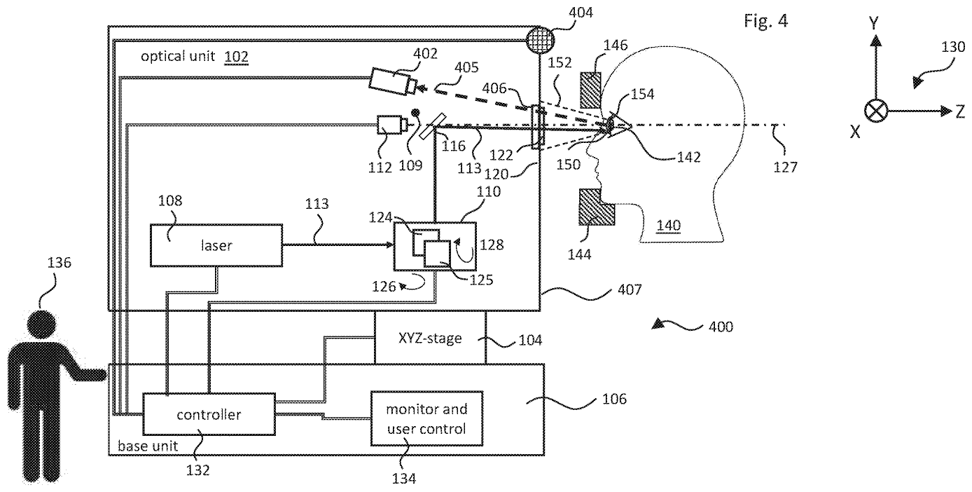

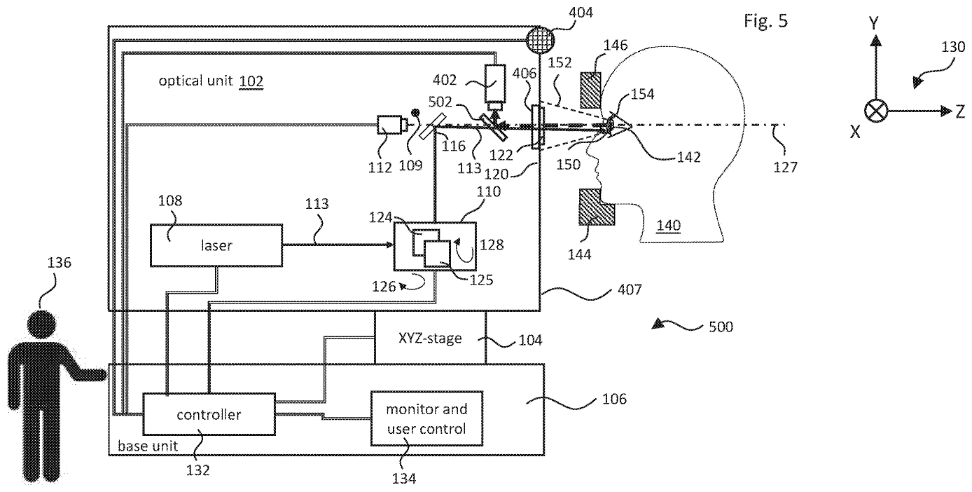

In an alternative embodiment, a contact lens, similar to lens 150 but without protrusions 162, may be used. In further embodiments, the TSCPC procedure may be carried out using apparatus 100 or the other apparatuses shown in FIGS. 4 and 5 without any contact lens. In these latter embodiments, controller 132 may operate scanner 110 to direct the laser beam to impinge on the treatment points without contact between any part of the apparatus and eye 142. In cases in which image processing is used to directly target the treatment points, the reference for locating the treatment points may be the outer limbal edge as described previously. Alternatively, the limbus itself may serve as the reference, as defined by various image processing algorithms, such as the location of the average pixel intensity value between the cornea and the sclera or the maximum gradient in pixel intensity values in these locations.

### Contact Lens Designs

FIGS. 2A, 2B, and 2C are schematic frontal views of three embodiments of contact lens 150 together with a view of eye 142 through the contact lens, in accordance with embodiments of the invention. Labels from FIG. 1 are used in FIGS. 2A-2C for identical or similar items.

The contact lens material, which is transparent at least to visible light and to laser beam 113, may comprise glass, plastic, or crystalline material, such as sapphire. It is advantageous that the refractive index of the material is close to that of conjunctiva 172 (1.38) and sclera 168 (1.41) so as to reduce refraction and reflection of light. For example, fused silica has a refractive index of 1.44 at 810 nm, and BK7 has a refractive index of 1.51.

FIG. 2A shows a frontal view of a contact lens 150A and eye 142. The frontal view of eye 142 shows pupil 178 in its center, surrounded by an iris 201, which in turn is surrounded by limbal ring 165, which is typically darker than the iris. The dark limbal ring 165 contrasts well against the white sclera 168, and therefore outer limbal edge 164 provides a good reference for aligning and sizing contact lens 150A to eye 142. Protrusions 162 may be evenly distributed on the circumference of a circle 202 (shown as a dotted line only for the purpose of clarity), which in a properly aligned lens 150A is concentric with edge 164 and pupil 178. Edge 164 typically has a diameter between 12 and 13 mm. A gap G between circle 202 of protrusions 162 and edge 164 is typically 1.5 mm, and may vary between 0.5 mm and 2 mm for alternative embodiments of lens 150A. Thus, the diameter of circle 202 is typically between 12.5 mm and 15 mm.

FIG. 2B shows a frontal view of a contact lens 150B, in which protrusions 162 are distributed on circle 202, but filling the circumference only partially: The protrusions form two arcs, a top arc 204 and a bottom arc 206, with gaps 208 and 210 between the arcs around respective positions of 3 o'clock and 9 o'clock. For example, contact lens 150B may be positioned on the eye so that arcs 204 and 206 cover respectively the superior and inferior quadrants of eye 142, with gaps 208 and 210 positioned respectively at the nasal and temporal quadrants of the eye.

FIG. 2C shows a frontal view of a contact lens 150C, in which protrusions 162 cover most of circle 202, but have a gap 212 around the 3 o'clock position. Lens 150C as shown in the figure is configured for treatment of the left eye, so that gap 212 extends over the temporal quadrant of the eye. The lens may be rotated by 180° for treatment of the right eye.

In the embodiments shown in FIGS. 2B and 2C, gaps 208 and 210 and gap 212, respectively, may be positioned over pterygium, heavily pigmented tissue, vasculature, nerves, or other sensitive anatomy, so as to avoid depressing or irradiating (and thereby irritating) such anatomy. Alternatively, the operator may designate which protrusions 162 not to irradiate.

In some embodiments, a contact lens, such as one of lenses 150A, 150B, or 150C, may comprise markings (such as lines, not shown in the figures) to facilitate identification of the protrusions in images captured by camera 112 and controller 132. The markings may be marked onto the surface of the lens or may comprise indentations in the lens. Each marking may be located at a predetermined distance from a respective one of the protrusions.

FIGS. 3A and 3B are schematic representations of two contact lenses 302 and 308 with different examples of profiles of protrusions 304 and 310, in accordance with embodiments of the invention.

FIG. 3A shows contact lens 302, which is similar to contact lens 150A of FIG. 2A, comprising protrusions 304. A partial sectional view 306 shows a cross-section of lens 302 at one of protrusions 304. Protrusions 304 have a spherical or dome-like profile, with a diameter of D₁ and a height of H₁.

FIG. 3B shows contact lens 308, which is also similar to contact lens 150A of FIG. 2A, comprising protrusions 310. A partial sectional view 312 shows a cross-section of lens 308 at one of the protrusions 310. Protrusions 310 have, unlike protrusions 304, a rectangular “top-hat” profile, with a diameter of D₂ and a height of H₂.

The typical diameter of a protrusion, such as diameters D₁ and D₂, is at least the diameter of beam 113 as it impinges on eye 142, e.g., at least 400 μm. Alternatively, the diameter may be larger, for example at least 600 μm, or even 800 μm. The typical height of a protrusion, such as heights H₁ and H₂, is at least 0.1 mm. Alternatively, the height may be larger, for example at least 0.5 mm, or possibly at least 0.7 mm or even 1.0 mm.

In alternative embodiments, the protrusions of the contact lens may have other profiles, as will be apparent to those skilled in the art after reading the present description.

In addition to facilitating alignment of laser beam 113 on the eye, the use of multiple small protrusions as in the present embodiments, rather than a larger continuous protrusion, is also advantageous in applying greater localized pressure, which increases the transmission of laser beam 113 into the eye at the target points. In alternative embodiments, however, the contact lens may comprise one or more arc-shaped protruding ridges.

In some embodiments, a kit of different contact lenses may be provided for use with system 100, such as two or more of the contact lenses shown in FIGS. 2A-2C and 3A-3B. The kit may comprise multiple contact lenses having different respective dimensions, such as different lens diameters and/or different heights and/or diameters of the protrusions. Furthermore, the lenses in the kit may have different respective patterns of protrusions and/or different diameters of circle 202 of protrusions, the latter providing different gaps G. Operator 136 may select a contact lens from the kit, for example, to match the diameter of outer limbal edge 164 or other properties of the eye.

### Monitoring the Procedure

FIG. 4 is a schematic side view of an automated TSCPC apparatus 400, in accordance with an alternative embodiment of the invention. Apparatus 400 is similar to apparatus 100 of FIG. 1, except for added thermal and acoustic feedback devices detailed hereinbelow. Labels from FIG. 1 are used in FIG. 4 for identical or similar items.

Apparatus 400 comprises, in addition to the components of apparatus 100, a thermal sensor, such as a thermal camera 402, and a microphone 404, both coupled to controller 132. Thermal camera 402 may comprise, for example, an Xi 400 Compact Industrial Imager with microscope optics, manufactured by Optris Infrared Sensing LLC (Portsmouth NH, USA). Thermal camera 402 captures a thermal image of eye 142 as indicated by an arrow 405. The field of view of thermal camera 402 is registered with the field of view of camera 112 so that, by tracking the position of eye 142 based on images from camera 112, controller 132 may map each portion of the thermal image to the precise portion of the eye from which the heat was emitted.

Optical unit 102 may comprise an enclosure 407 containing laser 108, scanner 110, camera 112, beam combiner 116, fixation point 109, and thermal camera 402. Enclosure 407 comprises a window 406 through which beam 113 of optical radiation is directed toward contact lens 150 and through which camera 112 and thermal camera 402 receive the image and the infrared radiation from eye 142. Window 406 is fabricated from a material that is transparent to visible, NIR (typical treatment beam), and IR wavelengths in a range extending into the mid-or long-IR, typically between at least 400 nm and 5000 nm or 8000-12000 nm. For example, the window may be fabricated from zinc selenide (ZnSe), zinc sulfide (ZnS), Cleartran®, or diamond.

In case the material of contact lens 150 is not transparent to IR wavelengths used for temperature monitoring, the contact lens may comprise through-holes for facilitating thermal imaging of the tissue surface, as further described hereinbelow.

For thermal feedback for the TSCPC procedure, thermal camera 402 senses the temperature of eye 142, based on received IR radiation, at each of the points contacted by protrusions 162. Controller 132 controls a dose of energy applied by laser 108 trough beam 113 at each of the points responsively to the temperature at the point. For example, when the temperature at a given point has reached a predetermined threshold temperature, laser beam 113 may be moved to the next target point or the laser parameters may be changed. The threshold temperature may be constant over an entire laser pulse, or changing (e.g., linearly increasing) in time during the pulse.

Microphone 404 receives acoustic signals during the procedure and transmits them to controller 132. The acoustic signals may include, for example, hisses and pops due to evaporation and cavitation of fluid irradiated by the laser beam. In one embodiment, the irradiation of ciliary body 174 continues until the ciliary body has been heated sufficiently to explode and produce an audible popping sound. Alternatively, the procedure may be controlled to avoid such tissue explosions. Thus, if a spike in the volume of the acoustic signal is received by microphone 404, it may indicate a “pop” from a tissue explosion, and may be used as a feedback signal to stop the laser irradiation at a given location. Additionally or alternatively, following one or more such acoustic spikes in volume and/or changes in the acoustic spectrum, controller 132 may analyze the signals captured by microphone 404 so as to identify a specific sound signal that preceded the spike, and may then designate this signal as an acoustic failure condition. This analysis may be performed using machine learning techniques or spectral-temporal analysis.

In an alternative embodiment, thermal camera 402 may have its own dedicated window for receiving IR radiation from the eye.

FIG. 5 is a schematic side view of an automated TSCPC apparatus 500, in accordance with another embodiment of the invention. Apparatus 500 is similar to apparatus 400 of FIG. 4, except that apparatus 500, the thermal image is captured by thermal camera 402 through a dichroic beamsplitter 502, which reflects the IR wavelengths used for temperature monitoring, while passing visible and near-IR wavelengths.

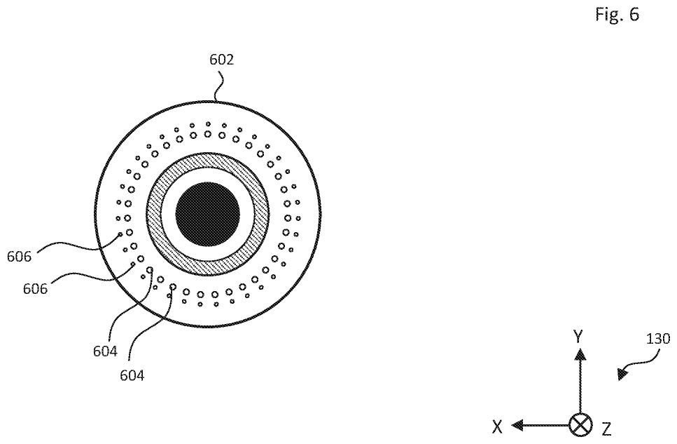

FIG. 6 is a schematic frontal view of a contact lens 602 with protrusions 604 and through-holes 606, in accordance with a further embodiment of the invention. Contact lens 602 is adapted specifically for use with a thermal sensor, such as thermal camera 402, in monitoring the temperature of the eye under treatment.

Contact lens 602 is similar to contact lens 150A (FIG. 2A), with details of eye 142 visible through the lens. In addition to protrusions 604 (similar to protrusions 162), through-holes 606 have been formed through lens 602, with each hole adjacent to a respective protrusion. Through-holes 606 are provided to enable mid-IR radiation to pass through lens 602 for thermal imaging, as described with reference to FIG. 4 hereinabove, when the material of lens 602 does not transmit the IR wavelengths used for thermal imaging. Holes 606 may each have a diameter smaller than, equal to, or larger than the typical diameter of protrusions 604. Each hole 606 is typically located within a distance of 0.2 mm from a respective protrusion 604.

### Method of Operation

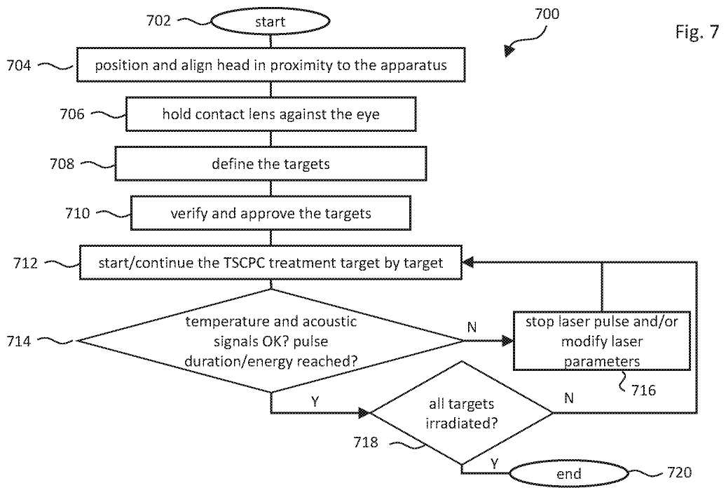

FIG. 7 is a flowchart 700 that schematically illustrates a method for performing a TSCPC procedure using TSCPC apparatus 400 (FIG. 4), in accordance with an embodiment of the invention.

The procedure starts in a start step 702. In a positioning step 704, operator 136 positions and aligns head 140 of the patient in front of apparatus 100 into chin rest 144 and forehead rest 146. In a contact lens step 706, contact lens 150 (or any of the other contact lenses supplied with the apparatus) is placed against eye 142, either by operator 136 or by eye-stabilizing structure 152. In a target definition step 708, operator 136 defines target points for the procedure on contact lens 150 in relation to protrusions 162. In a target verification step 710, operator 136 verifies and approves the location of the target points utilizing an image of eye 142 and contact lens 150 captured by camera 112.

In a treatment step 712, operator 136 activates the TSCPC treatment, causing laser 108 to fire a beam at one of protrusions 162. In a feedback step 714, controller 132 checks the thermal and acoustic feedback signals, as well as the laser pulse duration/energy. If the feedback signals indicate a problem, such as temperature exceeding a predefined threshold or an acoustic failure condition, or alternatively that a preset pulse duration or energy has not been reached, controller 132 stops the laser pulse. If required, controller 132 modifies the laser emission parameters in a modification step 716. The treatment parameters may be adjusted for the remaining target points. For example, the power and/or pulse duration may be reduced or increased. Alternatively or additionally, the temperature threshold may be adjusted downward or up. For example, the threshold may be set to the temperature that was observed slightly (e.g., 5-500 ms) before an acoustic failure condition. From step 716, the process returns to step 712 to treat the next target point.

If the feedback signals in step 714 do not indicate a problem, the process continues to a next target step 718, which checks whether all targets of the treatment plan have been irradiated. If the result is negative, the process continues to the next target at step 712, without passing through modification step 716, checking at each target again the feedback signals in step 714. (If no feedback is provided, such as in apparatus 100, steps 714 and 716 may be omitted.) After all targets have been irradiated (positive result in step 718,) the treatment stops at an end step 720.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A device for ophthalmic surgery, comprising:
a transparent material having a concave rear side, which is shaped to contact a front surface of an eye; and
multiple protrusions extending from the rear side and distributed azimuthally around the rear side.

2. The device according to claim 1, wherein the protrusions are configured to contact and depress respective points around a sclera of the eye.

3. The device according to claim 1, wherein multiple through-holes extend through the transparent material in respective proximity to the multiple protrusions.

4. The device according to claim 1, wherein the protrusions have a dome shape.

5. The device according to claim 1, wherein the protrusions have a rectangular profile.

6. The device according to claim 1, wherein the protrusions have a height of at least 0.1 mm.

7. (canceled)

8. The device according to claim 1, wherein the protrusions have a diameter of at least 0.4 mm.

9. (canceled)

10. The device according to claim 1, wherein the protrusions are distributed on a circle having a diameter between 12.5 and 15 mm.

11. The device according to claim 10, wherein the protrusions are distributed around an entire circumference of the circle.

12. The device according to claim 10, wherein the protrusions are distributed around a part of a circumference of the circle, with one or more gaps in the circumference in which there are no protrusions.

13. Apparatus for medical treatment, comprising:
a contact lens having a front side and a concave rear side, which is shaped to contact a front surface of an eye and comprises multiple protrusions extending from the rear side and configured to contact and depress respective points around a sclera of the eye;
a laser configured to generate a beam of optical radiation; and
a scanner, which is configured to direct the beam through the front side of the contact lens to impinge sequentially on at least some of the protrusions so as to photocoagulate tissue in the eye in proximity to the respective points.

14. The apparatus according to claim 13, and comprising:
a camera configured to capture an image of the contact lens in contact with the front surface of the eye; and
a controller, which is configured to process the image to identify the protrusions and to control the scanner to direct the beam to impinge on the protrusions.

15. The apparatus according to claim 14, and comprising a sensor, which is configured to sense a temperature of the eye at each of the points contacted by the protrusions, wherein the controller is configured to control a dose of energy applied by the laser at each of the points responsively to the temperature.

16. The apparatus according to claim 15, wherein the sensor is configured to sense infrared radiation emitted from the points.

17. The apparatus according to claim 16, and comprising an enclosure, which contains the scanner, the camera, and the sensor and which comprises a window through which the beam of optical radiation is directed toward the contact lens and through which the camera and the sensor receive the image and the infrared radiation from the eye, wherein the window is transparent in a range between 400 nm and at least 5000 nm.

18. The apparatus according to claim 17, and comprising a dichroic beamsplitter, which directs visible radiation emitted from the eye toward the camera and directs the infrared radiation emitted from the eye toward the sensor.

19. The apparatus according to claim 16, wherein the sensor comprises a thermal camera, which is configured to capture an infrared image of the eye.

20. The apparatus according to claim 16, wherein the contact lens has multiple through-holes extending therethrough in respective proximity to the multiple protrusions, wherein the sensor is configured to sense the infrared radiation that passes through the through-holes.

21. The apparatus according to claim 14, and comprising a microphone coupled to the controller and configured to receive acoustic signals from the eye while the beam is photocoagulating the tissue, wherein the controller is configured to control a dose of energy applied by the laser at each of the points responsively to the acoustic signals.

22. The apparatus according to claim 13, and comprising an eye-stabilizing structure, coupled between the contact lens and an optical unit containing the scanner.

23-48. (canceled)
